(19) United States
(12) Patent Application Publication
(10) Pub. No.: US 2005/0197858 A1
(43) Pub. Date: Sep. 8, 2005

(54) Web Enabled Image Extension System

(75) Inventor: Christopher Scott Lindsey, Jacksonville, FL (US)

Correspondence Address: CHRISTOPHER SCOTT LINDSEY, 12139 SPINEY RIDGE DRIVE SOUTH, JACKSONVILLE, FL, 32225, US

(73) Assignee: Mr. Christopher Scott Lindsey, Jacksonville, FL (US)

(21) Appl. No.: 10/708,336

(22) Filed: Feb. 25, 2004

(51) Int. Cl. A61B 8/14

(52) U.S. Cl. 705/2

(57) ABSTRACT

The Web Enabled Image Extension (WEIX) system provides extended access to patient medical scan images from the capabilities of traditional Computer Imaging systems. The WEIX system employs a Secure Web Server with add-on modules to provide a variety of user-level access. The WEIX system leverages this user-level access to create a type of workflow process to allow the diagnosis, dictation, transcription, and report release for medical scan procedures.

### BACKGROUND OF INVENTION

The field of Medical Imaging has been enhanced by the utilization of computer systems. However, the information obtained from Medical Imaging computer systems is not readily available outside of the immediate area; and typically requires expensive, proprietary equipment if remote viewing is even possible. In fact, most Medical Imaging modalities still rely on some form of Radiographic film for medical diagnosis and review. The Web Enabled Image Extension (WEIX) system will greatly extend the access and usability of Medical Imaging information in a secure and controlled manner. Also, the WEIX system will provide this information via common data formats available on most all Personal Computers (PCs) that have Internet Web-Browsing capability.

### SUMMARY OF INVENTION

The Web Enabled Image Extension (WEIX) System is a computer system that interfaces with existing medical scanning devices, such as Computed Tomography (CT) or Magnetic Resonance (MR) Imaging Systems, for the purpose of extending access to the image information collected in the CT and/or MR scans. The WEIX system is described in this document in terms of the particular functions of each of the major components.

### BRIEF DESCRIPTION OF DRAWINGS

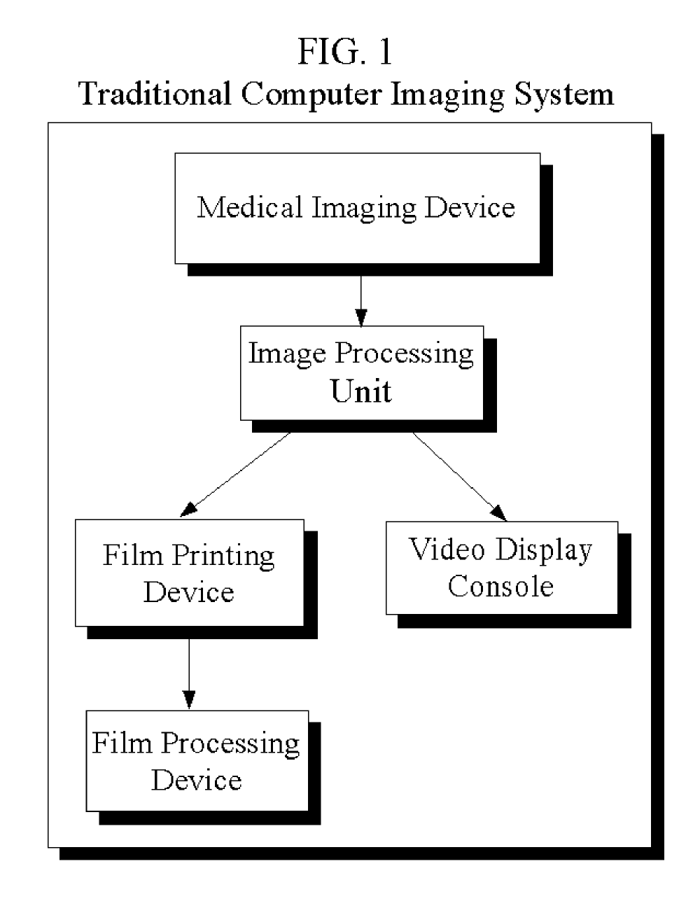

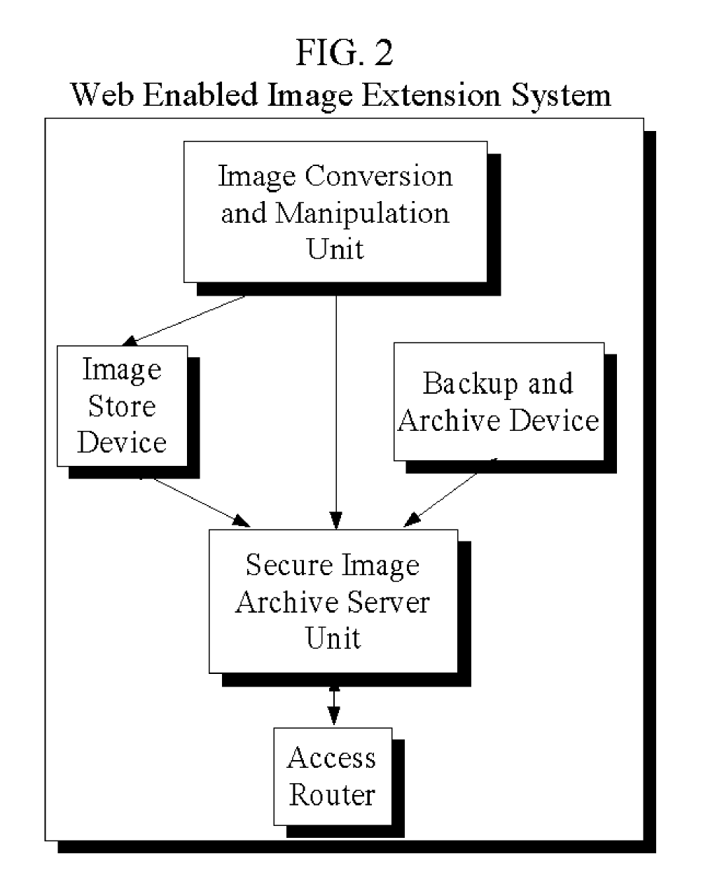

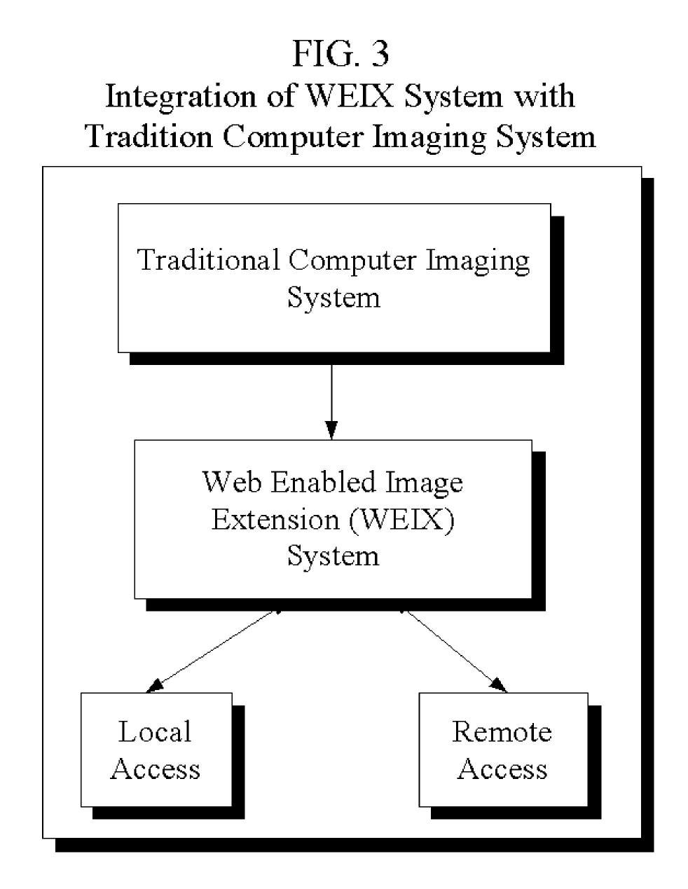

The drawing described as FIG. 1 (drawing sheet 1/3) demonstrates the information data flow between the major components in the traditional Computer Imaging System, such as the Computed Tomography (CT) and the Magnetic Resonance (MR) Imaging Systems. The drawing described as FIG. 2 (drawing sheet 2/3) shows the information data flow between the major components in the Web Enabled Image. The drawing described as FIG. 3 (drawing sheet 3/3) shows the information data flow from the traditional Computer Imaging System to the WEIX System; and the information data flow between the WEIX System and Local Access devices and/or Remote Access devices (i.e. computer workstations).

### DETAILED DESCRIPTION

The Medical Imaging Device (MID) and the Image Processing Unit (IPU) are the standard components of most all CT or MR Scanning Units. Please note that these are not the actual names for these devices; but rather a functional description of components. The IPU will require connectivity to the Image Conversion and Manipulation Unit (ICMU). It will be a requirement of the IPU to adopt an open standard of device connectivity, such as the Transmission Control Protocol/Internet Protocol (TCP/IP), Small Computer Systems Interface (SCSI), or Electronic Industries Association-232 (EIA-232/RS-232); or the IPU manufacturer will provide proprietary information regarding their recommended transport mechanism for connectivity to the ICMU.

Now assuming that TCP/IP will be used for connectivity, a file transfer mechanism (such as the File Transfer Protocol—FTP) procedure will be employed between the IPU and the ICMU. Since these devices will be connected in a point-to-point fashion; no additional security procedures are necessary other than limited physical access. The ICMU will have a port, or interface, configured to support the file transfer service in an Always-Ready receiving mode in order to accept unrequested file transfer data from the IPU. The IPU will be responsible for transferring the image files after each scanning procedure is completed. The file transfer process can be automatically initiated once the scanning procedure is done, or it can be done with operator intervention.

These image files relate to a single set of scan images that will be described as an Image Set. In addition to the scan images, the Image Set images sent from the IPU will include two special, informational files. The first file is a Start of Set (SOS) header file. The SOS file contains patient information about this Image Set. The second file is an End of Set (EOS) trailer file that contains the image file count and the file sizes of each file in the Image Set. The IPU will be responsible for creating both the SOS file and the EOS file.

The ICMU will have a spooler daemon responsible for collecting the Image Sets received from the IPU. This spooler daemon will verify the integrity of each Image Set by utilizing the information provided in the EOS file. It may be possible for the ICMU to acknowledge complete receipt of Image Sets; or to submit a Resend request to the IPU. This capability will require some type of handshaking mechanism between the IPU and the ICMU. Once the entire image set is received by the ICMU (that is, all scan images, the SOS file and the EOS file), the process continues.

The scan images contained in the Image Set are then manipulated by the ICMU and converted to a standard Lossless image format that can be viewed using standard web browsers. Lossless image formats are those image file types which maintain the original image data and do not result in any loss of image data during compression or file manipulation. Therefore, lossless image formats should provide the highest level of data integrity and quality, even during image compression and manipulation. The ICMU will save each of the newly created Lossless image files into the corresponding Image Set. The Lossless image formats are used to maintain integrity and image quality. Support for 3-Dimensional volumes and solids may be implemented as necessary. The standards for manipulation, printing, filming, and viewing of three dimensional (3-D) volumes and solids should be evaluated to assure image quality and integrity prior to a commitment of supportability. The recommendation for grayscale or black-white is Tagged Image File Format (TIFF); and the recommendation for color and/or grayscale is either Graphics Interchange Format (GIF) or Bit Map (BMP).

After processing by the ICMU, each filename in the Image Set is changed to identify the format utilized on the new, altered Image Set files. This identity of format types will facilitate multiple imaging formats (i.e. TIFF, GIF, and some 3-D volume format). Each of the scan images are altered into a standard image format type and then written to a Common Internet File System (CIFS) or Network File System (NFS) file system directory as a set entity, located on the Secure Image Archive Server (SIAS). The SOS and EOS files are written out to the SIAS archive files system as well. The communication path between the ICMU and the SIAS is of a point-to-point type and limited physical access to the SIAS should be adequate security. However, if security concerns demand it, Secure Shell (SSH) protocols can be employed (i.e. secure copy/scp or secure file transfer protocol/sftp). Also, an intelligent tree structure will be used to archive the Image Sets in a manner conducive to quick lookup and search tool.

At this point, the scan images in the Image Set have been converted into a standard image format type; and from this point onward the set will be described as Image Set′. It is to contain: an SOS file, image-1 to image-N, and an EOS file. Once the ICMU has completed processing an Image Set′, it will add an entry into a First-In First-Out Archive Queue (FIFO-AQ) that will serve as an acknowledgement to the SIAS that this Image Set′ is ready for processing. The FIFO-AQ will contain a pointer listing the location of this Image Set′ within the SIAS archive file system tree.

The SIAS has an archive daemon that will check every five minutes for the presence of new entries in the FIFO-AQ. This FIFO-AQ will be implemented as a linked-list; where each list entry contains a value that indicates the presence of a new Image Set′ that is waiting processing. The SIAS archive daemon scans the linked-list every five minutes to determine if the list is empty. If the list is empty, the daemon does nothing. However, if the list is not empty, the daemon processes each entry in the list. The steps performed for each entry are: (1) go to the head of the list and obtain the first entry; if the list is empty then quit; (2) use the entry's value to determine the pending Image Set′ location within the SIAS archive file system tree; and then load this value into a pointer variable; (3) confirm that the directory and Image Set′ files exist and confirm that this Image Set′ has not already been processed; (4) update the index of pointers to the Image Sets′ (utilizing either a database or a flat file); (5) if the web server will NOT utilize a database to facilitate dynamic queries to the index of pointers, then update the Hyper-Text Markup Language (HTML) listing of the index of pointers to the Image Sets′; (6) remove this entry from the list; (7) return to step 1 above. Note that the ICMU will only add entries to the FIFO-AQ and the SIAS will only remove entries from the FIFO-AQ.

A Hierarchical Storage Management (HSM) or a Storage Archive Management scheme may be utilized on the SIAS to assist in managing very large volumes of Image Set′ data. Otherwise, a backup strategy should be utilized to provide access to Image Sets′ that have been archived after some time of inactivity. Whatever method is employed to manage the Image Sets′ contained in the SIAS archive file system tree should meet the medical and legal requirements of the particular facility and/or organization.

The SIAS will support many methods of access to the Image Set′ data. Some, or all, of these methods can be utilized for each installation as deemed appropriate. The access methods will be contained in Modules. Some of the modules will be required and some will be optional.

The Printing Module is optional. It can be a part of the SIAS or it can be a separate system. This module will be responsible for formatting the images to be printed on paper. This formatting includes converted the images into a PostScript (PS) or Printer Control Language (PCL) format and sent to the printer. A networked, PostScript, printer with built-in PostScript (PS) support and Line Printer Daemon (LPD) support is suggested for printing. Note that standard filming capability will be provided by the existing CT/MR Scanner system.

The Web Server module will contain Secure Socket Layer (SSL) security; and will be a required module. The Web Server module will allow image retrieval and manipulation of two-dimensional images that can be viewed with any standard web browser capable of viewing images. The suggested image formats are Graphics Interchange Format (GIF) and Bitmap (BMP) since these formats should be viewable from most any browser. Secure access to the Web Server can be obtained via Local Area Network (LAN) connectivity (including Wireless), via Wide Area Network (WAN) connectivity utilizing a Virtual Private Network (VPN), and via the Internet. This connectivity will be provided via internetwork access routing devices (i.e. network router). The Web Server module can be contained in the SIAS, or it can be a standalone module on a separate system.

The SIAS Web Server will maintain a database of valid users that can access Image Set′ data. Once access is validated for a user, the user can access the HTML list of the index of pointers to select the Image Set′ desired for viewing. There will be thumbnails available as well as a selfguided slideshow of the Image Sets′. Image zooming and manipulation can be done within the standard web browser; in a similar fashion as done with map viewing tools found on popular web sites. Some image manipulation can also be accomplished by using standard image tools provided with the operating system of the computer workstation or personal computer. Paper printing of these images can be done using the standard printing capabilities of World Wide Web browsers, such as Netscape Communicator and Microsoft Internet Explorer.

Remote access via modem dial-in, or dial-back Challenge-Handshake Authentication Protocol (CHAP), can be provided for those who do not require Internet accessibility. Many CISCO routing devices provide this capability and can be utilized with computer workstations or personal computers (PC) to support this capability.

Virtual Private Network (VPN) access can be setup using a standard firewall with permitted access to the SSL port used (typically 443). The firewall will restrict all other TCP/IP traffic bound for the SIAS server. Also, the SIAS will permit SSL network traffic on port 443 only to those users with valid user accounts as setup in the Access Control List (ACL) utilized with the SIAS Web Server. Note: other authentication methods are acceptable, including one time passwords provided by Secure Token Cards. These secure token cards allow for a more secure and more restricted access.

The Web Server HTML page can be enhanced from the typical freestyle tabular listing of Image Sets′ if desired. An optional Web Portal can be incorporated to provide individualized, customized, views for each user or group of users. One can review Image Sets′, review diagnoses and interpretations to these Image Sets′, and possibly cross-reference older Image Sets′ for comparison. The portal can even be used to provide a queue of Image Sets′ awaiting diagnoses and interpretation. Likewise, the portal can be used to provide a queue of dictated interpretations waiting transcription or transcriptions waiting review and release. Also, the Web Server access can be utilized similar to a medical film library in order to allow Image Set′ review by the referring clinician.

The Diagnostic Module is also optional. It is made up of four optional sub-modules: the Dictation Module, the Transcription Module, Release Module, and the Permanence Module. The Diagnostic Module provides the capability of remote interpretation of CT/MR scan images. It provides for remote review of the Image Sets′, dictation of Image Sets′, transcription of dictation, review/release of transcriptions, and conversion of released transcriptions to a non-editable, viewable format

The Dictation Module can be utilized when remote dictation capability is needed. With this module, the client browser is setup to support launching an audio recording tool that can play and record standard audio file formats (such as wave files, .wav, or audio format, .au). Once the audio dictation file is completed; the client browser will upload the dictation file back to the SIAS server. These capabilities will be possible from the Uniform Resource Locator (URL) web page visible to those users with access permission to the dictation module URL (i.e. http://xxx.xxx.xxx/). The web server module will be responsible for placing this dictation audio file in the proper Image Set′ located on the SIAS archive file system. The web server module will also place an entry into a First-In First-Out Dictation Queue (FIFO-DQ). This entry will reference the respective Image Set′. It will serve as an indicator to the SIAS server that an audio file has been added to an Image Set′ and is waiting processing.

The SIAS will contain a dictation spooler daemon to process the FIFO-DQ. The FIFO-DQ entries will identify the particular dictation audio file that is ready to be forwarded on for transcription. The SIAS dictation spooler daemon will check every five minutes for the presence of new entries in the FIFO-DQ. This FIFO-DQ will be implemented as a linked-list; where each list entry contains a value that identifies an unprocessed Image Set′ audio file.

If the FIFO-DQ list is empty, the daemon does nothing. However, if the list is not empty, the daemon processes each entry in the list. The steps performed for each entry are: (1) go to the head of the list and obtain the first entry; if the list is empty then quit; (2) use the entry value to determine the Image Set′ location within the SIAS archive file system tree; and then load this value into a pointer variable; (3) confirm that the directory and Image Set′ files exist; and confirm that this Image Set′ audio file has not already been processed; (4) update an index of pointers to completed dictations that are pending transcription (utilizing either a database or a flat file); (5) if the web server will NOT utilize a database to facilitate dynamic queries of this index of pointers to dictation audio files, then update the Hyper-Text Markup Language (HTML) listing of the index of pointers to the dictation audio files pending transcription; (6) remove this entry from the list; (7) return to step 1.

The Dictation Module will generate a blank transcription text file entry for each Image Set′ that contains a completed dictation audio file. This module will generate an entry in a First-In First-Out Transcription Queue (FIFO-TQ). Each entry will list the location of the transcription text record within the SIAS archive file system. This transcription text record, or image, will not actually be ‘blank’, but rather it will contain required patient information formatted according to the requirements of the institution or corporation. Note that the FIFO-TQ entry will contain additional information that uniquely identifies the physician/clinician (PHID) who has performed the dictation. The PHID will be determined based on the user who submitted the dictation audio file for upload to the SIAS server above. If needed, the one providing the dictation can audibly include a second identification key within the actual dictation audio file.

The Transcription module will be responsible for the management of the transcription text record files. It will utilize the FIFO-TQ to provide an index of transcription records that need to be completed. This index can be a simple HTML file or a dynamically generated query. Access Control Lists (ACLs) will provide authorized users with read-write access to the transcription text documents; as well as read-only access to the corresponding audio dictation files for each Image Set′. The URL on the client system used by the transcriptionist(s) will launch a text writer program to allow editing of the incomplete, ‘blank’, transcription text files. The transcriptionist will complete the typing and sign this record with two digital signatures, one for the physician providing the dictation, and one for the transcriptionist. It will be imperative that the transcriptionist has simultaneous access to the dictation audio file and the corresponding transcription text file. The transcriptionist client system only requires an audio player capable of playing the audio dictation files and a text writer program capable of reading, writing, and appending of the transcription text files. A communication pathway (such as email) will be permitted to submit questions and concerns regarding unclear dictations. Once completed, the transcriptionist will upload the completed transcription text file via a submit capability available on their client browser's URL. Once the submitted transcription file is uploaded to the SIAS archive file system, an entry will be added to a First-In First-Out Release Queue (FIFO-RQ). The FIFO-RQ will provide the list of completed transcription records that are ready for review and final release.

The Release module will be responsible for management of final review of the transcription text files. This module will utilize the FIFO-RQ entries to create an index of pointers that list out the transcriptions that require final review and release of report. The Release module will notify the respective physicians or clinicians of transcriptions that need review. The notification can be provided via a portal URL entry (if a Portal is to be employed), or via an email which contains a hyperlink to the URL containing the transcription text file needing final review. Access to this URL will be provided via the standard ACL methodology as described above. The physician's client system will require the text writer application to facilitate reading and amending of the transcription text file. The physician will have read-write access to the transcription text file; as well as hyperlinks to the corresponding Image Set′ data (including audio dictation file and images) to allow final review prior to transcription release.

Once the physician reviews and approves the transcription text file, he/she will upload the file to the SIAS archive file system via a submit process. The transcription record is now ready for permanent release. The Release module will then create an entry in the First-In First-Out Permanence Queue (FIFO-PQ). The FIFO-PQ entries indicate transcription records that need to be converted to a permanent format.

The Permanence Module is responsible for converting the released transcription text files into a read-only formatted document, suitable for printing and archiving (such as PostScript or Portable Document Format, PDF). This module will utilize the FIFO-PQ to identify which transcription text files need to be converted to a permanent format.

This will provide some degree of integrity of reports for legal purposes. These PDF and/or PostScript files will be accessible to those users that have ACL access to the Image Set′ data via URL links.

The Permanence Module will utilize a permanence spooler daemon to monitor the FIFO-PQ. The Permanence spooler daemon scans the linked-list every five minutes to determine if the list is empty. If the list is empty, the daemon does nothing. However, if the list is not empty, the daemon processes each entry in the list. The steps performed for each entry are: (1) go to the head of the list and obtain the first entry; if the list is empty then quit; (2) use the entry value to determine the Image Set′ location within the SIAS archive file system tree; and then load this value into a pointer variable; (3) confirm that the directory and Image Set′ files exist; and confirm that this Image Set′ transcription text file has not already been processed; (4) convert the transcription text file to PostScript and/or Portable Document Format and place these transciptionX.ps and transcriptionX.pdf files into the correct location within the SIAS archive file system; (5) update the index of pointers to Image Sets′ (utilizing either a database or a flat file); (6) if the web server will NOT utilize a database to facilitate dynamic queries of this index of pointers, then update the Hyper-Text Markup Language (HTML) listing of the index of pointers to Image Sets′; (7) remove this entry from the list; (8) remove the transcription text file; (9) return to step 1. Note that the dictation audio files and transcription text files will not be accessible to any user except the assigned physician (or physician group) and the assigned transcription (or transcription group).

To support cross-referencing old Image Sets′, a database may be employed for search capabilities. Otherwise, a simple search scheme can be used to search the entire SIAS archive file system tree of patient data. Image Sets' may be placed in offline access, which may require complex retrieval schemes such as provided with the HSM or SAM software.

The Monitor Module is responsible for monitoring the health of the entire WEIX system. It will interface will all other installed modules to provide a daily HTML report for each module. The Monitor module will utilize a Monitor daemon to perform this capability. The pages will be in HTML format and will be indexed for viewing. ACL will be utilized to restrict access to the reports to only those needing to monitor the WEIX system. If a transport mechanism is available (such as email), the reports can be forwarded to a monitoring agent or service for review. Any noted problems or errors can be used initiate the repair procedure, possibly using a dial-in, or call-back, remote access. The HTML reports will include the following types: error logs, access logs, system logs, and status logs.

The Access Control Module will be used for management of the ACLs used in the WEIX system. A Lightweight Directory Access Protocol (LDAP) style of hierarchy will be used to logically group the various type of user access needed for each module. Managing user accounts should be done with secure access in mind. For temporary user access, such as with a Virtual Film Library Checkup, a guest account that expires within one week could be used. For remote users, one time passwords (as provided with Secure Token Cards) is probably best. Password aging should be considered to maintain proper access. Authentication should be considered carefully to restrict all access to a per user level.

Requests for additions and deletions of users can be done via electronic mail, Email, (if email transport exists), or by using a text message form on a URL web page. This communication is to be kept available for recording purposes. Telephone, email, or onsite correspondence can be used to setup users with the initial connectivity to the WEIX system. An orientation class could be used to provide password maintenance recommendations and a training course of the WEIX system. Types of authorization to various components of the WEIX system can be discussed at the orientation meeting.

1. A method for extending the viewing capability of current, traditional medical computer imaging systems to a standard world wide web browser for authorized users; without the need for a digitizing device or elaborate computer workstation system.

2. A method for implementing a Filmless and Paperless medical scan procedure which complements the current, traditional Medical Computer Imaging systems.

3. A method for providing the capabilities of: remote diagnosis, remote dictation, remote transcription, and remote report release of images/studies performed with current, traditional Medical Computer Imaging systems by standardizing the formats and protocols used for these tasks to standards and protocols used in standard World Wide Web browsers.

4. A method for providing controlled, private access to images/studies and corresponding reports of current, traditional Medical Computer Imaging systems by utilizing secure World Wide Web access over the Internet; or by utilizing circuit-switched, dial-up, or leased communication lines.
